(19) United States
(12) Patent Application Publication
(10) Pub. No.: US 2004/0191343 A1
(43) Pub. Date: Sep. 30, 2004

(54) Hepatoprotective pharmaceutical composition comprising a mixture of coumarinolignoids, process for preparation thereof

(76) Inventors: Sunil Kumar Chattopadhyay, Uttar Pradesh (IN); Sachin Srivastava, Uttar Pradesh (IN); Arvind Singh Negi, Uttar Pradesh (IN); Archana Gupta, Uttar Pradesh (IN); Suman Preet Singh Khanuja, Uttar Pradesh (IN)

Correspondence Address: NIXON & VANDERHYE, PC, 1100 N GLEBE ROAD, 8TH FLOOR, ARLINGTON, VA, 22201-4714, US

(21) Appl. No.: 10/402,140

(22) Filed: Mar. 31, 2003

(51) Int. Cl. A61K 35/78; A61K 31/366

(52) U.S. Cl. 424/776; 514/452

(57) ABSTRACT

The present invention relates to a pharmaceutical composition useful as a hepatoprotective agent, said composition comprising of a mixture of coumarinolignoids of formula 1,2 and 3. The present invention also relates to a improved process for production of liver protective composition containing coumarinolignoids of formula 1, 2 and 3 from Cleome viscosa with optimized ratios of the same. More particularly, the present invention relates to a processing technology for the isolation of a combination of coumarinolignoids Cliv-92 comprising a mixture of coumarinolignoids of formula 1,2 and 3 from the seeds of Cleome viscosa.

### FIELD OF INVENTION

The present invention relates to a pharmaceutical composition useful as a hepatoprotective agent, said composition comprising of a mixture of coumarinolignoids of formula 1, 2 and 3. The present invention also relates to a improved process for production of liver protective composition containing coumarinolignoids of formula 1,2 and 3 from Cleome viscosa with optimized ratios of the same. More particularly, the present invention relates to a processing technology for the isolation of a combination of coumarinolignoids Cliv-92 comprising a mixture of coumarinolignoids of formula 1, 2 and 3 from the seeds of Cleome viscosa. More particularly, the present invention relates to a processing technology for the preparation of a combination of three coumarinolignoids of formula 1, 2 and 3 in a ratio of 3:5:2 from the seeds of the plant Cleome viscosa.

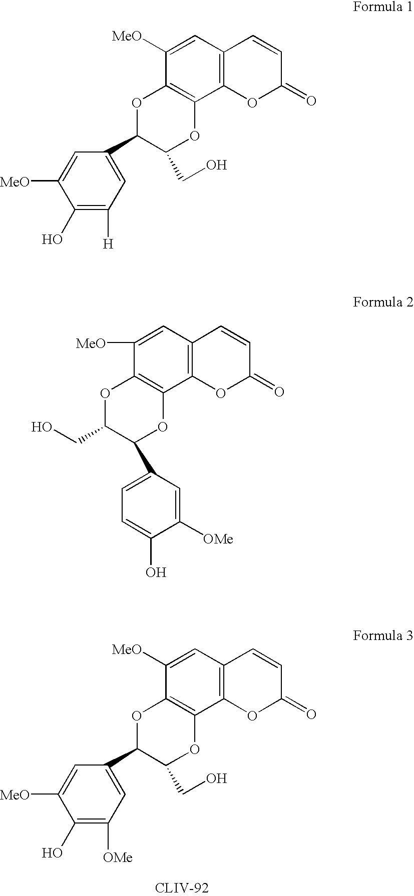

### BACKGROUND OF THE INVENTION

In India liver diseases is the third killer of humankind next to cardiovascular and cancer. Liver is a vital organ for metabolism and excretion and storage. The disorders of the liver will be classified as hepatitis (inflammation of the liver), chronic hepatitis, hepatosis (non-inflammatory disorders) and liver cirrohosis. Furthermore, the detoxification of a numbers of drugs and xenobiotics occurs in the liver. The bile secrected by the liver has among other things an important role in digestion. Most hepatoxic chemicals damage liver cells mainly by inducing lipid peroxidation and other oxidative damage. Enhanced lipid peroxidation produced during liver microsomal metabolism of ethanol results in hepatitis and cirrohosis. The majority of cases of acute hepatitis are due to viruses. Hepatitis B infection often results in chronic liver diseases and cirrohosis of liver. Primary liver cancer has been shown to be developed by these viruses. It has been reported that approx. 14-16 million people are infected with hepatitis viruses in south east Asia and about six percent of the total population of the region are carrier of the virus. Despite the tremendous advances made in medicine, no effective liver protective agent is yet available. Plant derived drugs are known to play a vital role in the management of liver diseases. About 6% of (3.6 billion US $) of the total herbal drug business (approx. 60 billion US $) is for hepatic diseases.

Cleome viscosa is an annual herb, which occurs as a weed in cultivated as well as in rain fed soils from North east to Northern parts of India. In a screening program for the isolation of antihepatotoxic compounds from higher plants, we have found that a combination of three compounds from the seeds of Cleome viscosa possesses significant liver protective activity. The combination of these three compounds has been designated by us as Cliv-92. Cliv-92 is a combination of three closely related coumarinolignoids of formula 1, 2 and 3. Coumarinolignoids are a novel class of natural products in which a lignan (C6C3 unit) is linked with a coumarin moiety through a dioxane bridge.

Previously, we have standardized a processing technology in which Cliv-92 was obtained in a ratio of 7:2:1 (Indian patent No. 182638 and 182637). Now, we have improved upon our previous process and developed an improved process in which the combination of the above coumarinolignoids was obtained in a ratio of 3:5:2. Cliv-92 in a ratio of 3:5:2 exihibit better antihepatotoxic activity as compared to its ratio of 7:2:1 which is comparable or even better than silymarin, the standard drug that is currently being used for the treatment of liver diseases.

Our previous process of isolation of Cliv-92 involved extracting the air-dried pulverized seeds with aliphatic solvent at room temperature, extracting the defatted material with alcohol at room temperature and concentrating the solvent to a residue, adsorbing the above residue with a suitable adsorbent and extracting the adsorbed material with aromatic hydrocarbon and chlorinated solvent and isolationg Cliv-92 from the above fractions.

The disadvantages of the above process included extraction of the adsorbed material with chlorinated solvents which did not extract the coumarinolignoids exhaustively and resulted in lower yields of the coumarinolignoids. Also, the disadvantage of the above process includes that Cliv-92 was obtained in a ratio of 7:2:1. Biological testing of Cliv-92 in different ratio revealed that it exhibited potent liver protective activity in which the ratio of the three coumarinolignoids are in a ratio of 3:5:2.

### OBJECTS OF THE INVENTION

The main object of the present invention is to provide a pharmaceutical composition useful as a hepatoprotective agent comprising of a mixture of coumarinolignoids of formula 1, 2 and 3 from Cleome viscosa.

It is another object of the invention to provide an improved process for the production of a combination of coumarinolignoids of formula 1, 2 and 3 from Cleome viscosa with optimized ratios of the same.

Another object of the present invention is to develop a processing technology in which the above three coumarinolignoids Cliv-92 can be isolated in a ratio of 3:5:2 for showing potent antihepatotoxic effects.

Still another object of the present invention is to develop a processing technology in which the combination of the three compounds could be isolated in higher yields.

Still another object of the present invention is to develop a green processing technology in which no toxic chemicals are used for isolation of the coumarinolignoids from the seeds of Cleome viscosa.

Yet another object of the present invention is to develop a processing technology for isolation of Cliv-92 in a cost-effective way.

Still another object of the present invention is to develop a processing technology for isolation of Cliv-92 on large scale for commercial production.

### SUMMARY OF THE INVENTION

Accordingly the present invention provides a pharmaceutical composition useful as a hepatoprotective agent, said composition comprising of a mixture of coumarinolignoids of formula 1, 2 and 3 in a ratio of 3:5:2 respectively, along with a pharmaceutically acceptable carrier

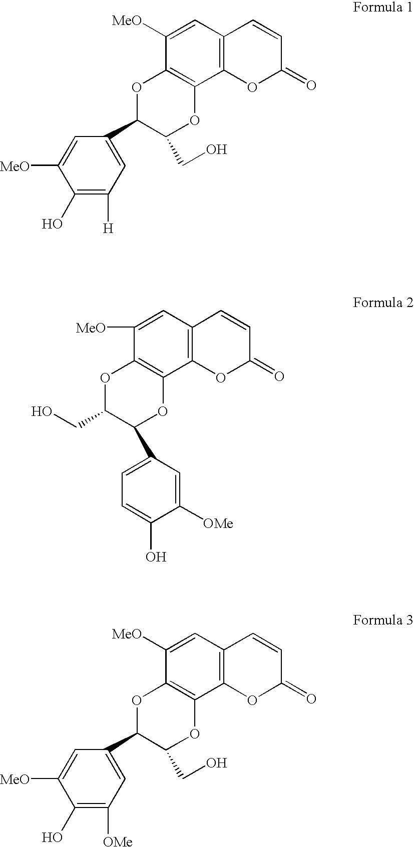

The present invention also provides an improved process for the production of a liver protective pharmaceutical composition comprising of a mixture of coumarinolignoids of formula 1, 2 and 3 in a ratio of 3:5:2 respectively from the seeds of Cleome viscosa

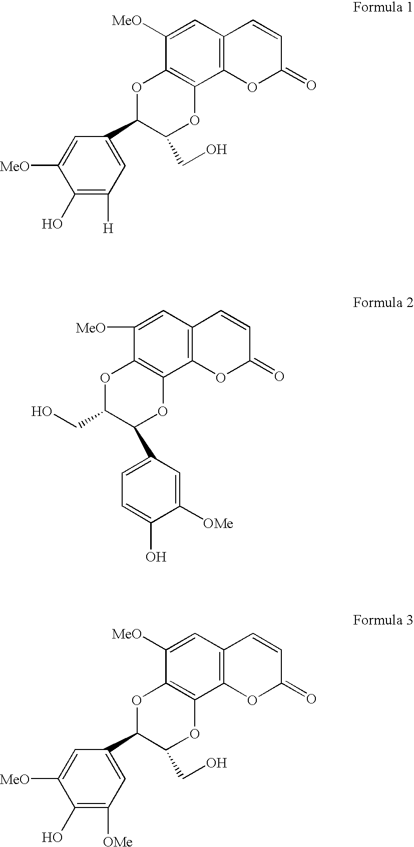

said process comprising extracting the dried and pulverized seeds of Cleome viscosa with an aliphatic solvent at a temperature in the range of 20-40° C. to obtain defatted seeds, (ii) extracting the defatted seeds with alcohol at a temperature in the range of 20-40° C. and concentrating the solvent to obtain an alcoholic extract, (iii) adsorbing the alcoholic extract with a suitable adsorbent and drying the adsorbed material at a temperature in the range of 20-50° C. for 4-80 hours, (iv) extracting the adsorbed material with an aromatic hydrocarbon solvent at a temperature in the range of 100-130° C. for 8-48 hours followed by ethyl acetate at a temperature in the range of 50-90° C. for 8-48 hours and (v) concentrating the solvents from the above fractions by filtration to obtain the a filtrate containing a composition comprising a mixture of coumarinolignoids of formula 1, 2 and 3 and (vi) subjecting the filtrate to chromatography for additional yield of coumarinolignoids of formula 1, 2 and 3, in a ratio in the range of 7:2:1 to 1:7:2 respectively.

In one embodiment of the invention, the aliphatic solvent is selected from petroleum-ether and hexane, preferably petroleum-ether (60-80° C.).

In another embodiment of the invention, the alcohol comprises an alkanol selected from ethanol and methanol.

In a further embodiment of the invention, the suitable adsorbent material is selected from the group comprising of celite, cellulose and a mixture thereof, preferably celite.

In a further embodiment of the invention, the adsorbed material is dried at 30° C. for 60 hours.

In another embodiment of the invention, the aromatic hydrocarbon solvent is selected from toluene and toluene-pet-ether (60-80° C.)(1:1), preferably toluene.

In yet another preferred embodiment of the invention, the adsorbed material was extracted with aromatic hydrocarbon at 125° C. for 48 hours.

In another embodiment of the invention, the adsorbed material was extracted with ethyl acetate at 85° C. for 48 hours.

In another embodiment of the invention, filtration was performed using ethyl acetate, acetone, toluene, methanol and a mixture of toluene and ethyl acetate (3:1).

In a further embodiment of the invention, chromatography was done using silica gel, silicic acid, florosil and eluting with petroleum-ether (60-80° C.), petroleum-ether (60-80° C.)-ethylacetate (1:1) mixture and ethyl acetate successively.

In a preferred embodiment of the invention, silica gel was used for chromatography.

### DETAILED DESCRIPTION OF THE INVENTION

The present invention provides a process for the production of an antihepatotoxic composition Cliv-92 which is a combination of three closely related coumarinolignoids of formula 1, 2 and 3 in a ratio of 3:5:2 from the plant Cleome viscosa by extracting the dried and pulverized seeds The extraction is carried out using an aliphatic solvent and preferably at a temperature in the range of 20-40° C. to defat the seeds. The defatted seeds are then extracted with alcohol, preferably at a temperature in the range of 20-40° C. and the solvent concentrated to obtain an alcoholic extract. The alcoholic extract is adsorbed with a suitable adsorbent and the adsorbed material then dried preferably at a temperature ranging from 20-50° C. for 4-80 hours. The adsorbed material is then extracted with an aromatic hydrocarbon solvent at a temperature ranging from 100-130° C. for 8-48 hours followed by extraction with ethyl acetate at a temperature ranging from 50-90° C. for 8-48 hours. The solvents are then concentrated from their respective fractions to obtain Cliv-92 by filtration. The filtrate is subjected to chromatography for additional yield of Cliv-92.

The aliphatic solvent is preferably selected from petroleum-ether and hexane, preferably petroleum-ether (60-80° C.). The alcohol used is an alkanol selected from ethanol and methanol. The adsorbent material is preferably selected from celite, cellulose and a mixture thereof, preferably celite. The adsorbed material is dried at 30° C. for about 60 hours.

The aromatic hydrocarbon solvent used is selected from toluene and toluene-pet-ether (60-80° C.)(1:1), preferably toluene. The adsorbed material is extracted with the aromatic hydrocarbon preferably at 125C for about 48 hours and then extracted with ethyl acetate at 85° C. for about 48 hours. Filtration of the fractions after concentration of the fractions obtained after extracting was performed using ethyl acetate, acetone, toluene, methanol or a mixture of toluene and ethyl acetate (3:1).

The filtrate obtained was further subjected to chromatography using silica gel, silicic acid or florosil and then eluted with petroleum-ether (60-80° C.), petroleum-ether (60-80° C.)-ethylacetate (1:1) mixture and ethyl acetate successively.

In a preferred embodiment of the invention, silica gel was used for chromatography.

The invention is described in detail in the examples given below which are illustrative, and, therefore, should not be construed to limit the scope of the invention.

### EXAMPLE 1

Air dried and pulverized seeds (7 Kilograms) of C. viscosa were defatted by percolation at room temperature with petroleum-ether (60-80° C.) (10 litres×3) for four days. The defatted material was then exhaustively extracted with methanol (10×5 litres) for seven days. The methanolic solution of the extract was concentrated to a residue and adsorbed with celite (2 Kgs) and dried at 30° C. for 60 hours. The adsorbed material was packed in a filter paper thimble and extracted with toluene (5 litres) at 125° C. for 48 hours and then with ethyl acetate (5 litres) at 85° C. for 48 hours successively. The above two fractions on concentration furnished Cliv-92 which was collected by filtration with methanol. Total yield obtained was 6.5 grams. The filtrate was concentrated and chromatographed over silica gel in petroleum ether. The column was eluted with pet.ether-ethyl acetate (1:1) (3 litres) and with ethyl acetate (4 litres) successively. The above two fractions on concentration crystallized out and filtered with toluene-ethyl acetate (1:1) to give Cliv-92 (0.5 gram).

### EXAMPLE 2

Air dried and pulverized seeds (7 Kilograms) of C. viscosa were defatted by percolation at room temperature with hexane (10 litres×3) for three days. The defatted material was then exhaustively extracted with ethanol (10×5 litres) for five days. The ethanolic solution of the extract was concentrated to a residue and adsorbed with cellulose-celite (1:1) mixture (2 kgs) and dried at 30° C. for 60 hours. The adsorbed material was packed in a filter paper thimble and extracted successively with toluene-pet. ether (60-80° C.) (5 litres) at 100° C. for 48 hours and then with ethyl acetate (5 litres) at 85° C. for 48 hours. The above two fractions on concentration furnished Cliv-92 which was collected by filtration with toluene-ethyl acetate (1:1) mixture. Total yield obtained was 6.5 grams. The filtrate was concentrated and chromatographed over florosil in petroleum ether. The column was eluted with pet. ether-ethyl acetate (1:1) (3 litres) and with ethyl acetate (4 litres) successively. The above two fractions on concentration crystallized out and filtered with toluene-ethyl acetate (1.1) to give Cliv-92 (0.5 gram).

### Advantages

1. The extraction process described in this invention does not use any extreme conditions of temperature and pressure and is thus adaptable to commercial production of Cliv-92.

2. Solvents used in extraction process can be recycled and thus the process is cost effective.

3. No toxic chemicals and solvents have been used in the process of the invention and thus the process is ecofriendly.

4. No water partitioning is used to isolate Cliv-92 in the process and thus the process is suitable for large scale isolation of the compound and cost effective.

5. The use of solid matrix (celite, cellulose etc) adsorption technique helps to isolate Cliv-92 in a straight forward way with high yields.

We claim:

1. A pharmaceutical composition useful as a hepatoprotective agent, said composition comprising of a mixture of coumarinolignoids of formula 1, 2 and 3 in a ratio of 3:5.2 respectively, along with a pharmaceutically acceptable carrier

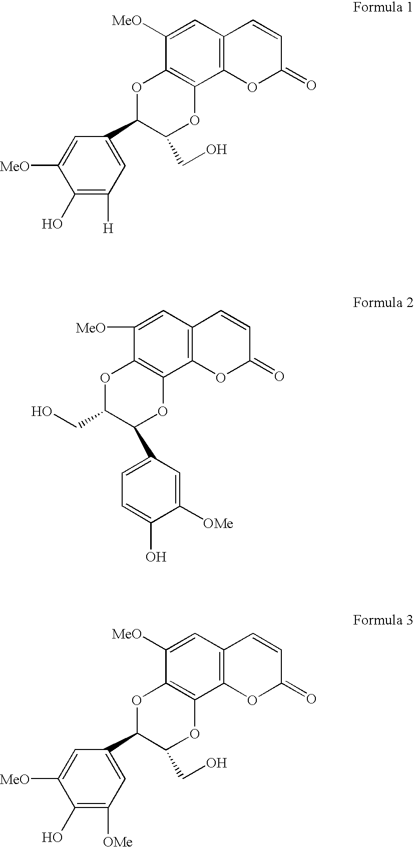

2. A process for the production of a liver protective pharmaceutical composition comprising of a mixture of coumarinolignoids of formula 1, 2 and 3 in a ratio of 3.5:2 respectively from the seeds of Cleome viscosa 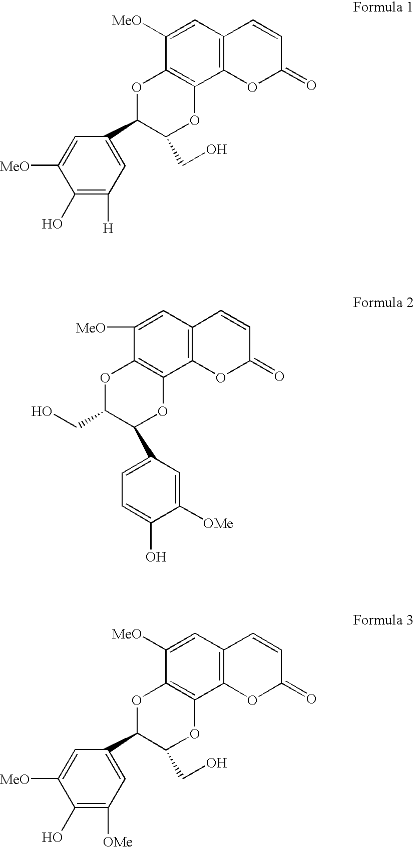
said process comprising extracting the dried and pulverized seeds of Cleome viscosa with an aliphatic solvent to obtain defatted seeds, (ii) extracting the defatted seeds with alcohol and concentrating the solvent to obtain an alcoholic extract, (iii) adsorbing the alcoholic extract with a suitable adsorbent and drying the adsorbed material, (iv) extracting the adsorbed material with an aromatic hydrocarbon solvent followed by ethyl acetate and (v) concentrating the solvents from the above fractions by filtration to obtain the a filtrate containing a composition comprising a mixture of coumarinolignoids of formula 1, 2 and 3 and (vi) subjecting the filtrate to chromatography for additional yield of coumarinolignoids of formula 1, 2 and 3, to obtain a final composition comprising coumarinolignoids of formula 1, 2 and 3.

3. A process as claimed in claim 2 wherein the extraction with aliphatic solvent is carried out at a temperature in the range of 20-40° C.

4. A process as claimed in claim 2 wherein the aliphatic solvent is selected from petroleum-ether and hexane.

5. A process as claimed in claim 4 wherein the aliphatic solvent is petroleum-ether (60-80° C.).

6. A process as claimed in claim 2 wherein the alcoholic extraction is carried out at a temperature in the range of 20-40° C.

7. A process as claimed in claim 2 wherein the alcohol comprises an alkanol selected from ethanol and methanol.

8. A process as claimed in claim 2 wherein the suitable adsorbent material is selected from the group comprising of celite, cellulose and a mixture there of.

9. A process as claimed in claim 8 wherein the suitable adsorbent is celite.

10. A process as claimed in claim 2 wherein the drying of the adsorbed material is carried out at a temperature in the range of 20-50° C. for 4-80 hours.

11. A process as claimed in claim 10 wherein the adsorbed material is dried at a temperature of 30° C. for 60 hours.

12. A process as claimed in claim 2 wherein the aromatic hydrocarbon solvent is selected from toluene and toluene-pet-ether (60-80° C.)(1:1).

13. A process as claimed in claim 12 wherein the aromatic hydrocarbon is toluene.

14. A process as claimed in claim 2 wherein extraction of adsorbed material with aromatic hydrocarbon is carried out at a temperature in the range of 100-130° C. for 8-48 hours.

15. A process as claimed in claim 14 wherein the adsorbed material is extracted with aromatic hydrocarbon at 125° C. for 48 hours.

16. A process as claimed in claim 2 wherein the adsorbed material was extracted with ethyl acetate at a temperature in the range of 50-90° C. for 8-48 hours.

17. A process as claimed in claim 16 wherein the adsorbed material was extracted with ethyl acetate at 85° C. for 48 hours.

18. A process as claimed in claim 2 wherein the of the concentrated fractions is carried out using ethyl acetate, acetone, toluene, methanol or a mixture of toluene and ethyl acetate in a ratio of 3:1.

19. A process as claimed in claim 2 wherein the chromatography is carried out using silica gel, silicic acid or florosil followed by elution with petroleum-ether (60-80° C.), petroleum-ether (60-80° C.)-ethylacetate (1:1) mixture and ethyl acetate successively.

20. A process as claimed in claim 19 wherein the chromatography is carried out using silica gel.

21. A process as claimed in claim 2 wherein the solvents used are recycled.

22. A process as claimed in claim 2 wherein the use of water partitioning to isolate the hepatoprotective composition is avoided.

23. A process as claimed in claim 2 wherein the combination of the three coumarinolignoids of formula 1, 2 and 3 respectively is in a ratio in the range of 7:2:1 to 1:7:2 for showing liver protective activity.

24. A process as claimed in claim 15 wherein the ratio of the combination of the three coumarinolignoids of formula 1, 2 and 3 is 3:5:2 respectively.
